(19) United States
(12) Patent Application Publication
(10) Pub. No.: US 2010/0305428 A1
(43) Pub. Date: Dec. 2, 2010

(54) ULTRASONIC GUIDANCE OF SUBCUTANEOUS TUNNELING

(75) Inventors: Matthew Bonner, Plymouth, MN (US); Robert Stadler, Shoreview, MN (US)

Correspondence Address: MEDTRONIC, INC., 710 MEDTRONIC PARKWAY NE, MINNEAPOLIS, MN, 55432-9924, US

(73) Assignee: MEDTRONIC, INC., Minneapolis, MN (US)

(21) Appl. No.: 12/512,652

(22) Filed: Jul. 30, 2009

Related U.S. Application Data
(60) Provisional application No. 61/182,309, filed on May 29, 2009.

(51) Int. Cl. A61B 8/00

(52) U.S. Cl. 600/424

(57) ABSTRACT

A method of tracking a medical device in a body includes inserting the medical device into a body and emitting a sonic signal from a transceiver coupled to the medical device. The method further includes receiving an echo of the sonic signal with the transceiver and detecting a location of the medical device in the body based on the received echo.

### CROSS-REFERENCE TO RELATED APPLICATIONS

This application claims the benefit of U.S. Provisional Application No. 61/182,309, filed on May 29, 2009. The entire disclosure of the above application is incorporated herein by reference.

### FIELD

The present disclosure relates to subcutaneous tunneling and, more particularly, to a tool for subcutaneous tunneling having ultrasonic guidance capability and a method for using the same.

### INTRODUCTION

Certain medical devices are introduced into a patient's body and extend from one area in the body to another. These devices can be introduced by forming a subcutaneous tunnel in the body of the patient and advancing an implantable medical component through the tunnel. Other medical components, such as electrical leads are similarly introduced into the patient's body through tunnels. More specifically, a tunneling tool is inserted into the body, and as the tunneling tool advances within the body, the tool creates a tunnel by partially separating a subcutaneous layer from the muscular plane. The medical component, such as a lead of a cardiac device (e.g., an implantable cardioverter-defibrillator device or pacing device), a catheter, a shunt, a neural stimulating or recording device, or a drug pump component, can be pulled by the tool through the tunnel as the tunneling tool creates the tunnel.

Typically, at least in the case of a cardiac lead, the medical professional advances the tunneling tool from the lateral side of the patient, toward the patient's back, and toward the patient's spine. Care is taken to ensure that the leading end of the tunneling tool is maintained at a proper depth within the body as it advances such that the leading end is unlikely to move toward or pierce the outer surface of the skin and/or move deeper in the body to pierce muscle or penetrate the pleural space. For instance, medical professionals can often see or feel a bulge in the skin as the tool advances in the patient, and the bulge indicates the position of the leading end of the tool. Thus, the medical professional monitors the bulge to maintain the tunneling tool at a proper depth and/or to realize when the tool is at its final position adjacent the patient's spine.

However, this procedure is usually performed while the patient is lying supine on a table. Thus, the table can interfere with the medical professional's ability to see or feel the tool through the patient's skin, especially as the tool moves along the back toward the patient's spine.

Other devices are extended into the body without first creating a tunnel with a tunneling tool. For instance, some catheters can be introduced into the body through a small incision, and the catheter can then be advanced further into the body through the incision, leaving a proximal end of the catheter outside the body. Once a distal end of the catheter is in a predetermined target position, leads, probes, and other components can be moved through the catheter to the predetermined target location.

The following discloses various exemplary embodiments of a device and a method for extending medical instruments into a patient's body in a convenient and accurate manner.

### SUMMARY

This section provides a general summary of the disclosure, and is not a comprehensive disclosure of its full scope or all of its features.

A method of tracking a medical device in a body is disclosed. The method includes inserting the medical device into a body and emitting a sonic signal from a transceiver coupled to the medical device. The method further includes receiving an echo of the sonic signal with the transceiver and detecting a location of the medical device in the body based on the received echo.

In another aspect, a method of creating a subcutaneous tunnel in a body is disclosed. The method includes inserting a leading end of a tunneling tool into a body and creating the subcutaneous tunnel as the leading end advances in the body. The method also includes emitting a sonic signal from an emitter on the tunneling tool and receiving an echo of the sonic signal. The method further includes detecting a location of the leading end in the body based on the received echo.

In still another aspect, a tracking system is disclosed that includes a medical device for being introduced into a body of a patient. The system also includes a transceiver that emits a sonic signal and that receives an echo of the sonic signal. The transceiver is coupled to the medical device. Furthermore, the system includes a controller that detects a location of the medical device in the body based on the echo received by the transceiver.

Moreover, a tunneling system for creating a subcutaneous tunnel in a body is disclosed. The tunneling system includes a tunneling tool having a leading end that creates the subcutaneous tunnel as the leading end advances in the body. The tunneling system also includes an emitter that emits a sonic signal, wherein the emitter is coupled to the tunneling tool. The system additionally includes a receiver that receives an echo of the sonic signal. The tunneling system further includes a controller that detects a location of the leading end in the body based on the echo received by the receiver.

In still another aspect, a method of creating a subcutaneous tunnel in a body is disclosed. The method includes coupling an electrical lead of a cardiac-defibrillator to a tunneling tool. The method also includes inserting a leading end of the tunnel tool into a body and creating the subcutaneous tunnel as the leading end advances in the body. The method further includes advancing the electrical lead in the tunnel. Moreover, the method includes emitting at least one ultrasonic signal from an emitter coupled adjacent the leading end and receiving a plurality of echoes of the ultrasonic signal. The method includes detecting a depth of the leading end within the body based on a reflection of the ultrasonic signal from the lung and a skin-air interface. Moreover, the method includes detecting a distance of the leading end from a predetermined target location based on a reflection of an ultrasonic signal from a spinal column. Additionally, the method includes detaching the tunneling tool from the electrical lead when the leading end is located approximately at the predetermined target location. Also, the method includes removing the tunneling tool from the body.

Further areas of applicability will become apparent from the description provided herein. The description and specific examples in this summary are intended for purposes of illustration only and are not intended to limit the scope of the present disclosure.

### DRAWINGS

The drawings described herein are for illustrative purposes only of selected embodiments and not all possible implementations, and are not intended to limit the scope of the present disclosure.

FIG. 1 is a front view of an exemplary embodiment of a tracking system with a tunneling tool according to various teachings of the present disclosure;

FIG. 2 is a detail view of the tunneling tool of FIG. 1 and an attached electrical lead;

FIG. 3 is a perspective view of the tunneling tool shown during use;

FIG. 4 is a sectional view of the tunneling tool and a body taken along the line 4-4 of FIG. 3;

FIG. 5 is a schematic view of a tracking system for the tunneling tool of FIG. 1;

FIG. 6 is a flowchart illustrating a method of using the tunneling tool of FIG. 1; and

FIG. 7 is a sectional view of the tracking system according to various other embodiments of the present disclosure.

Corresponding reference numerals indicate corresponding parts throughout the several views of the drawings.

### DETAILED DESCRIPTION

Exemplary embodiments will now be described more fully with reference to the accompanying drawings.

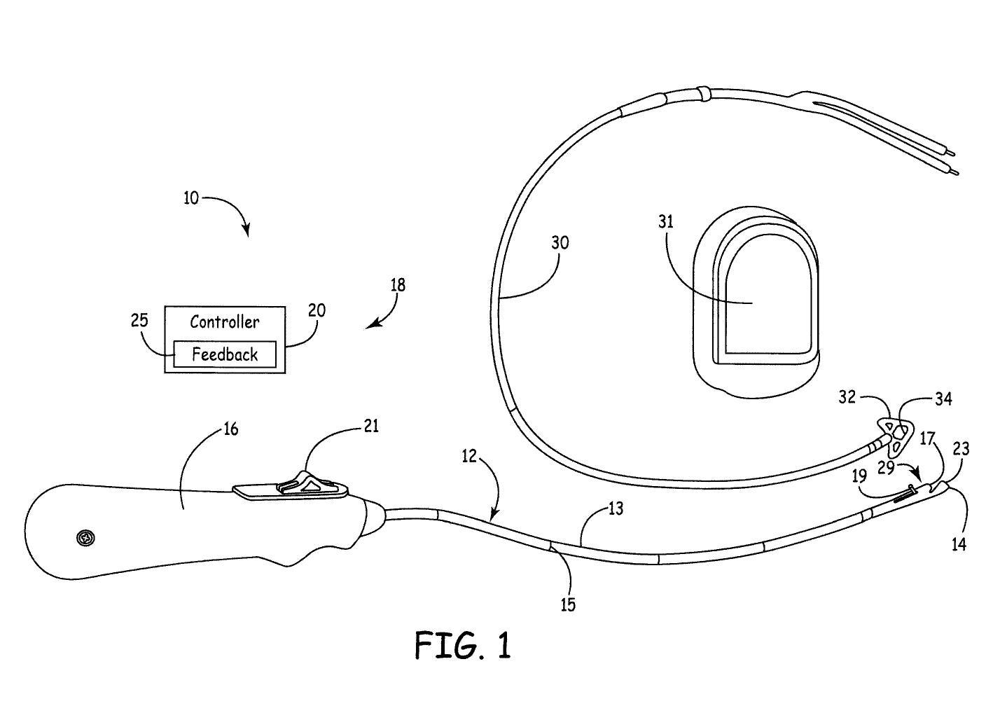

Referring initially to FIG. 1, a tracking system 10 is illustrated according to various exemplary embodiments of the present disclosure. The tracking system 10 generally includes a medical device, such as a tunneling tool 12. In some exemplary embodiments, the tunneling tool 12 can be Tunneling Tool Model 6996T, commercially available from Medtronic, Inc. of Minneapolis, Minn., or of a type discussed in U.S. Patent Publication Nos. 2008/0208339, 2008/0208303, 2008/0208248, and 2008/0208247, each to Rutten et al., or U.S. patent application Ser. No. 12/250,670 to Wengreen et al., each of which is hereby expressly incorporated by reference in its entirety. As will be discussed in greater detail below, the tracking system 10 can be used to track (i.e., locate) the tunneling tool 12. However, it will be appreciated that the tracking system 10 can be used for tracking any suitable medical device other than the tunneling tool 12. For instance the tracking system 10 can track a catheter, a neurological stimulating or recording device, a shunt, or an electrical lead in some exemplary embodiments.

In the exemplary embodiment of FIG. 1, the tunneling tool 12 can include a main body 13, which can be a slightly curved, elongate rod. The main body 13 can be made out of any suitable material, such as stainless steel. Also, the main body 13 can include a plurality of length indicators 15, such as bands that included at known intervals along the length of the main body 13. As will be discussed, the main body 13 can be inserted within a patient's body to form a tunnel therein, and the indicators 15 can provide the user with a general indication of how far the main body 13 has been extended into the patient's body.

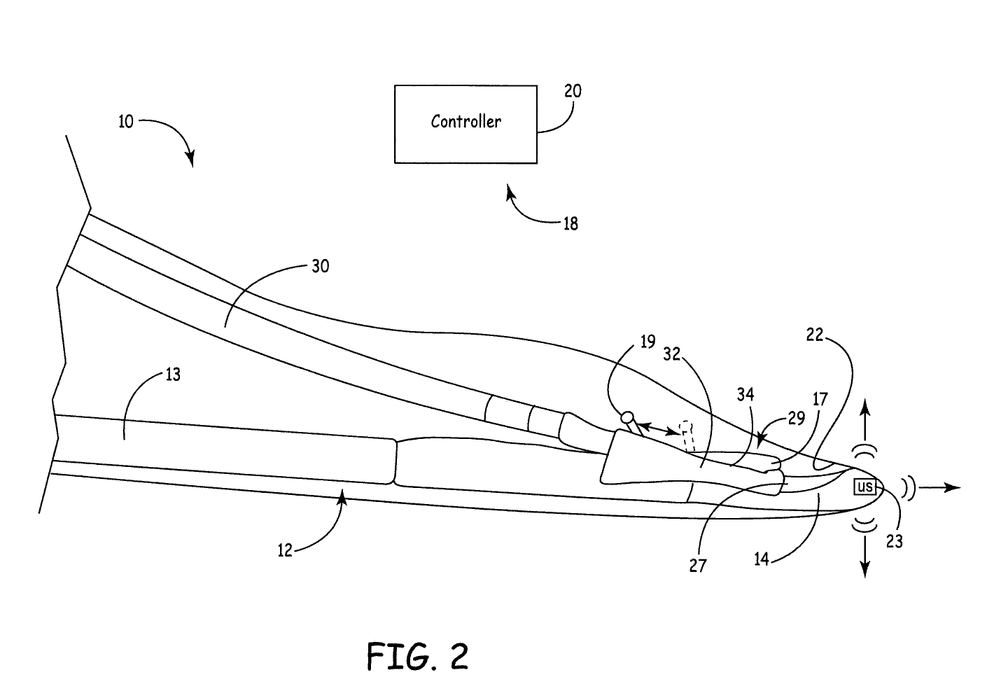

As shown in FIGS. 1 and 2, the tunneling tool 12 can include a leading end 14. As shown in FIG. 2, the leading end 14 can be rounded. As will be discussed, the leading end 14 can create a subcutaneous tunnel as the leading end 14 advances through the patient's body.

The tunneling tool 12 can also include a handle 16 at an end opposite from the leading end 14. The handle 16 can be made out of any suitable material, such as a polymeric material. The handle 16 can be ergonomically designed and shaped according to a user's hand. For instance, the handle 16 can include recesses for facilitating gripping of the handle 16. Thus, as will be described, a user can grasp the handle 16 and advance the leading end 14 through the patient's body.

As shown in FIG. 2, the tunneling tool 12 can also include an attachment mechanism, generally indicated at 29. In some embodiments, the attachment mechanism 29 includes a first hook 17 and a second hook 19. More specifically, the first hook 17 can extend into a recess 27 formed adjacent the leading end 14. The first hook 17 can extend generally parallel to the axis of the main body 13. Also, the second hook 19 can extend generally transverse to the axis of the main body 13. As shown in FIG. 2, the second hook 19 can be movable relative to the main body 13. For instance, the second hook 19 can move in a direction generally parallel to the axis of the main body 13. As shown in FIG. 1, the tunneling tool 12 can include an actuator button 21 or slider that is operatively coupled to the handle 16, and the actuator button 21 can be operatively coupled to the second hook 19 via a cable (not shown) that extends through the main body 13. Thus, the user can move the second hook 19 between a first position (shown in phantom in FIG. 2) and a second position (shown in solid lines in FIG. 2) by manipulating the actuator button 21. Accordingly, the actuator button 21 allows the user to selectively move the second hook 19 relative to the main body 13.

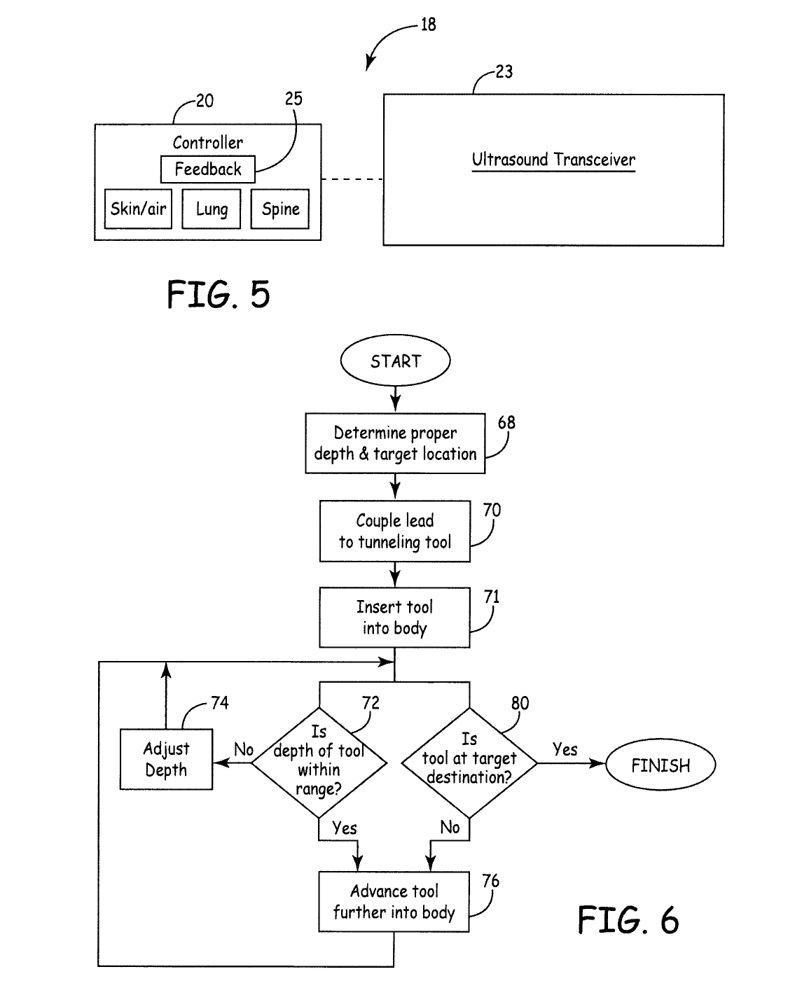

As shown in FIGS. 1-5, the tracking system 10 can also include a sonar system 18. As will be discussed, the sonar system 18 can emit sonic signals, which can reflect from various features of the patient's anatomy. These echoes can be received by the sonar system 18 in order to detect a location of the leading end 14 of the tunneling tool 12 relative to the patient's anatomy.

As shown in FIGS. 1-5, the sonar system 18 can include a signal transceiver 23 that emits the sonic signal and receives echoes of those sonic signals. In some embodiments, the signal transceiver 23 can include one or more piezoelectric crystals that emit and receive ultrasonic signals. However, it will be appreciated that the signal transceiver 23 can include any type of sonic emitter/receiver utilizing any suitable sonic signal. It will also be appreciated that although the signal transceiver 23 can be both a sonic emitter and receiver, the sonar system 18 can include a separate sonic emitter and receiver without departing from the scope of the present disclosure. The transceiver 23 can be coupled at a predetermined distance away from the leading end 14 of the tunneling tool 12.

The sonar system 18 can also include a controller 20. The controller 20 can be included in a computer, such as a laptop or desktop computer. The controller 20 can also include a feedback system 25, such as a visual display, an audible alarm, a tactile feedback device (e.g., vibration motor) and the like. The controller 20 can be in communication with the signal transceiver 23. For instance, the controller 20 can be in wireless communication with the signal transceiver 23 to receive information therefrom. The controller 20 can process the information received from the transceiver 23 to detect the location of the leading end 14 of the tunneling tool 12 within the patient. Then, as will be described, the controller 20 can utilize the feedback system 25 to thereby indicate the location of the tunneling tool 12.

As shown in FIG. 1, a separate medical device, such as an electrical lead 30 of a cardiac defibrillator or pacing device 31, can be coupled to the tunneling tool 12. For instance, in some exemplary embodiments, the electrical lead 30 can be a Subcutaneous Lead System Model 6996 SQ, which is commercially available from Medtronic, Inc. of Minneapolis, Minn., or of a type discussed in U.S. Patent Publication Nos. 2008/0208339, 2008/0208303, 2008/0208248, and 2008/0208247, each to Rutten et al., or U.S. patent application Ser. No. 12/250,670 to Wengreen et al., each of which is hereby expressly incorporated by reference in its entirety. As will be discussed, the tunneling tool 12 can be used to form a tunnel within a patient for passage of the electrical lead 30.

It will be appreciated that the tracking system 10 can be associated with any suitable cardiac device or any other medical device without departing from the scope of the present disclosure. For instance, the tracking system 10 can be associated with a catheter for implanting a neural stimulator, a neural recording device, a drug pump, and the like.

Specifically, as shown in FIG. 1, an exemplary embodiment of the lead 30 can include a distal end 32 with an opening 34. The distal end 32 of the lead 30 can be coupled to the leading end 14 of the tunneling tool 12 via the attachment mechanism 29. Specifically, with the second hook 19 in the first position (shown in phantom in FIG. 2), the opening 34 in the lead 30 can receive both the first and second hooks 17, 19 of the tunneling tool 12. Then, the user can manipulate the actuator button 21, thereby pulling the second hook 19 away from the first hook 17 and toward its second position (shown in solid lines in FIG. 2). As a result, the end 32 of the lead 30 can be stretched and held between the first and second hooks 17, 19 to be secured to the tunneling tool 12.

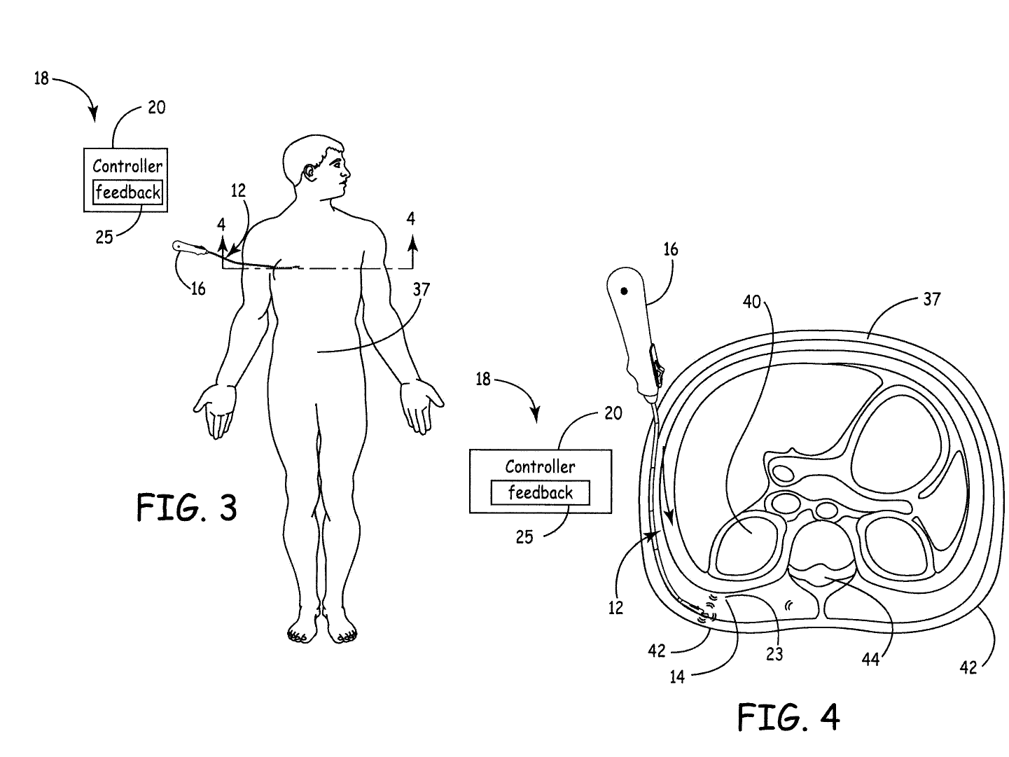

Once the lead 30 is attached, the user can insert the leading end 14 of the tunneling tool 12 and the attached lead 30 into a prepared incision in the patient 37 (FIGS. 3 and 4). The user then advances the leading end 14 toward the back of the patient 37. It will be appreciated that as the leading end 14 advances within the patient 37, the leading end 14 separates the patient's skin from the patient's muscle and/or fat, creating the subcutaneous tunnel 22 (FIG. 2). Also, as the leading end 14 advances, the tunneling tool 12 pulls and advances the lead 30 within the tunnel 22.

Furthermore, as the leading end 14 advances within the patient 37, the signal transceiver 23 can emit one or more ultrasound signals. The ultrasound signal(s) can reflect off of (i.e., create an echo on) various anatomical features of the patient 37. For instance, as illustrated in FIG. 4, the ultrasonic signal(s) can reflect from the lung 40 of the patient 37, from the skin-air interface (i.e., the outer surface of the skin of the patient 37), and/or the spinal column 44 (e.g., one or more vertebrae) of the patient 37 The signal transceiver 23 can receive one or more of these echoes, and upon receiving these echoes, the signal transceiver 23 can transmit a corresponding signal to the controller 20. The controller 20 can process this signal in order to determine the distance from the leading end 14 relative to the lung 40, the skin-air interface 42, and/or the spinal column 44 of the patient 37.

It will be appreciated that any suitable anatomical feature other than the lung 40, the skin-air interface 42, and the spinal column 44 can be relied upon for reflecting the ultrasonic signal and for detecting the position of the leading end 14 relative to the patient 37. For instance, the ultrasonic signal can echo off plural rib bones to determine the relative position of the leading end 14 from an area between the rib bones.

It will be appreciated that the transceiver 23 can emit a single ultrasonic signal, and the transceiver 23 can receive resultant echoes reflecting off of each of the lung 40, the skin-air interface 42 and/or the spinal column 44. Also, it will be appreciated that the controller 20 can distinguish between the echoes of the ultrasonic signal from the lung 40, the skin-air interface 42, and the spinal column 44. For instance, the controller 20 can distinguish between these signals based on the different frequencies of each, based on the timing of each, or in any other suitable manner.

In other embodiments, the transceiver 23 can emit and receive separate signals for respective ones of the lung 40, the skin-air interface 42, and the spinal column 44 (e.g., by separate piezoelectric crystals positioned according to the respective anatomy). Thus, in some embodiments, an ultrasonic signal can be directed toward the lung 40 and an echo can be received therefrom. Then, an ultrasonic signal can be transmitted toward the skin-air interface 42, and an echo can be received therefrom. Subsequently, an ultrasonic signal can be transmitted toward the spinal column 44, and an echo can be received therefrom. As stated above, the controller 20 can distinguish between each of the echoes.

Furthermore, the tunneling tool 12 can include a plurality of transceivers 23 that are disposed at predetermined locations on the leading end 14. For instance, the leading end 14 can include a transceiver 23 disposed generally toward the skin-air interface 42, which transmits signals toward and receives echoes from the skin-air interface 42, to thereby detect the distance between the leading end 14 and the skin-air interface 42. Likewise, the leading end 14 can include a separate transceiver 23 disposed generally toward the lung 40, which transmits signals toward and receives echoes from the lung 40, to detect the distance between the leading end 14 and the skin-air interface 42. Furthermore, the leading end 14 can include another separate transceiver 23 disposed generally toward the spinal column 44, which transmits signals toward and receives echoes from the spinal column 44, to detect the distance between the leading end 14 and the spinal column 44. Accordingly, the tunneling tool 12 can be advanced through the patient in a predetermined orientation such that the individual transceivers 23 are disposed generally toward the skin-air interface 42, lung 40, and spinal column 44, respectively. The transceivers 23 can be each dedicated for detecting echoes from separate anatomy to facilitate classification of the different echoes and to increase accuracy of the procedure.

Thus, as will be discussed in greater detail, the controller 20 can detect the depth of the leading end 14 within the patient 37 based on the relative distance of the leading end 14 from the lung 40 and/or the skin-air interface 42. Also, assuming that the user preselects a target location of the leading end 14 (i.e., a point at which to stop advancing the leading end 14 within the patient) relative to the spinal column 44, the controller 20 can detect a distance of the leading end 14 from the preselected target location.

Moreover, the feedback system 25 can provide feedback to the user relating to the location of the leading end 14. For instance, the feedback system 25 can visually display the current location of the leading end 14 within the patient. Also, the feedback system 25 can provide an audible or tactile alarm if the leading end 14 moves too close to the skin-air interface 42 (i.e., too shallow) and/or if the leading end 14 moves too close to the lung 40 (i.e., too deep). Moreover, the feedback system 25 can provide such an alarm when the leading end 14 reaches the predetermined target distance from the spinal column 44 (i.e., the leading end 14 has reached its predetermined target location).

Referring now to FIG. 6, an exemplary embodiment of a method of operation of the tracking system 10 is illustrated. Beginning in step 68, the user can determine a proper depth for the leading end 14 of the tunneling tool 12 as it advances through the patient 37. Step 68 can also include determining a final target location of the leading end 14 (i.e., a point at which the user should stop advancing the leading end 14). As stated above, the predetermined depth and/or target location can be determined relative to the anatomy of the patient 37. For instance, the user can use X-ray, CAT scans, or any other suitable imaging technology to determine the location of the patient's lung 40, skin-air interface 42, and/or spinal column 44. The user can also determine where the leading end 14 should be located relative to this anatomy to form the subcutaneous tunnel 22. The information acquired in step 68 can be stored in computerized memory within the controller 20.

Next, in step 70, the user couples the lead 30 of the defibrillator device 31 to the leading end 14 of the tunneling tool, as discussed above. Then, in step 71, the user inserts the tunneling tool 12 into the patient 37 and begins advancing the leading end 14 within the patient 37.

Then, the sonar system 18 can simultaneously detect the depth of the leading end 14 and the distance of the leading end 14 away from the target location within the patient 37. More specifically, in decision block 72, it is determined whether the depth of the leading end 14 is within a predetermined range (i.e., whether the leading end 14 is within a predetermined distance range from both the lung 40 and the skin-air interface 42). The controller 20 can compare the actual depth of the leading end 14 to the predetermined depth acquired in step 68 in decision block 72. If the leading end 14 is too shallow (i.e., too close to the skin-air interface 42), step 74 follows. Likewise, if the leading end 14 is too deep (i.e., the leading end 14 is too close to the lung 40), then step 74 follows.

In step 74, the feedback system 25 can indicate to the user that the leading end 14 needs to be changed in depth. As a result, the user can manipulate the handle 16 to change the depth of the leading end 14 and bring the leading end 14 within the preselected range of acceptable depths. Decision block 72 follows step 74, and it is again determined whether the leading end 14 is at the proper depth within the patient 37.

Assuming the leading end 14 is within the preselected range of acceptable depths (i.e., decision block 72 answered affirmatively), step 76 follows. In step 76, the user further advances the leading end 14 into the patient 37. Then, decision block 72 follows, and the system continues to monitor whether the leading end 14 is at the proper depth.

As stated, the sonar system 18 can also continuously detect whether the leading end 14 is located at the target location within the patient 37 in decision block 80. The controller 20 can compare the actual location of the leading end 14 relative to the spinal column 44 to the predetermined target location acquired in step 68. If the system 18 determines that the leading end 14 has not arrived at the target location (i.e., decision block 80 answered negatively), the feedback system 25 provides a representative feedback signal, and step 76 follows. Thus, the user knows that it is safe to further advance the leading end 14 in the patient 37 toward the target location.

Once the leading end 14 has arrived at the target location (i.e., decision block 80 answered affirmatively), the feedback system 25 indicates this fact to the user. Thus, the user knows to stop advancing the tunneling tool 12 within the patient 37.

Then, the user can detach the tunneling tool 12 from the electrical lead 30 by manipulating the actuator button 21 such that the second hook 19 moves toward the first hook 17. As such, the first and second hooks 17, 19 can be removed from the end 32 of the lead 30, and the tunneling tool 12 can be removed from the patient 37 by pulling the tunneling tool 12 in a reverse direction through the subcutaneous tunnel 22.

Accordingly, the tracking system 10 enables the user to quickly and conveniently form a subcutaneous tunnel 22. Also, the sonar system 18 allows the user to track the location of the tunneling tool 12 for forming the tunnel 22 in a very accurate manner. Additionally, it will be appreciated that the transceiver 23 can be coupled directly to any other suitable medical device other than the tunneling tool 12 for tracking purposes. For instance, the transceiver 23 can be coupled to a catheter, a neurological stimulating or recording device, a shunt, an electrical lead for a cardiac device, or any other suitable device, and the tracking system 10 can track the associated medical device as described above.

Furthermore, in some exemplary embodiments, the tracking system 10 is used to determine an individual patient's anatomical characteristics. More specifically, the tracking system 10 can determine the distance between the skin-air interface 41 and the lung 40 for an individual patient 37 while advancing through the patient 37, and the feedback system 25 can help the user to maintain the leading end 14 at a predetermined location between the skin-air interface 41 and the lung 40 while the leading end 14 advances. For instance, the feedback system 25 can be used to maintain the leading end 14 about equidistant from both the lung 40 and the skin-air interface 41. As such, separate imaging technologies (e.g., X-ray machines, CAT scans) may not be necessary for determining the anatomical features of the patient 37 and for determining a predetermined depth and/or target destination for the leading end 14 prior to use of the tracking system 10.

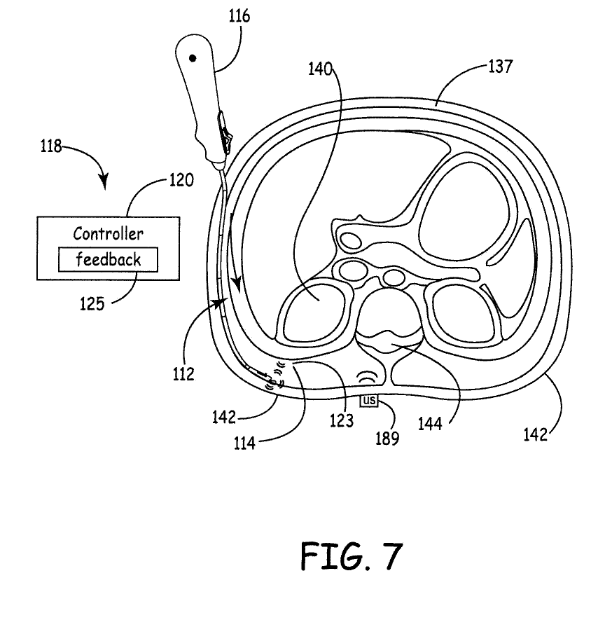

Referring now to FIG. 7, another exemplary embodiment of the tracking system 110 is illustrated. Components that are similar to exemplary the embodiments of FIGS. 1-6 are identified with corresponding reference numerals increased by 100.

As shown, the system 110 can include an external sonic device 189. The external sonic device 189 can be of any suitable device, such as one or more piezoelectric crystals that emit(s) an ultrasonic device. The external sonic device 189 can be disposed outside the patient 137 at a predetermined, known location. For instance, the external sonic device 189 can be adhered to the patient's skin, over the target location (i.e., predetermined destination) for the leading end 114. More specifically, in some embodiments, the external sonic device 189 can be attached to the patient 137 adjacent the spinal column 144. The external sonic device 189 can also be in communication with the controller 120. It will be appreciated that the system 110 can include a plurality of external sonic devices 189.

During operation, the external sonic device 189 can emit an ultrasonic signal. This signal can be received by the signal transceiver 123 on the tunneling tool 112, and the controller 120 can process this signal to detect the location of the leading end 114. The controller 120 can triangulate the location of the leading end 114, for instance, if there are multiple external sonic devices 189. More specifically, the controller 120 can detect the timing between transmission and receipt of the ultrasonic signal to detect the location of the leading end 114. As such, the signal from the transceiver 123 can act as a reference location such that the location of the leading end 114 can be detected. Accordingly, the distance of the leading end 114 from the target location can be detected.

In other exemplary embodiments, the external sonic device 189 merely receives the sonic signal(s) emitted from the signal transceiver 123 of the tunneling tool 112 so that the location of the leading end 114 can be detected. In still other embodiments, the external sonic device 189 emits a sonic signal toward the leading end 114, and the sonic signal reflects off of the leading end 114 to be received again by the external sonic device 189 to detect the location of the leading end 114.

The foregoing description of the embodiments has been provided for purposes of illustration and description. It is not intended to be exhaustive or to limit the invention. Individual elements or features of a particular embodiment are generally not limited to that particular embodiment, but, where applicable, are interchangeable and can be used in a selected embodiment, even if not specifically shown or described. The same may also be varied in many ways. Such variations are not to be regarded as a departure from the invention, and all such modifications are intended to be included within the scope of the invention.

Exemplary embodiments are provided so that this disclosure will be thorough, and will fully convey the scope to those who are skilled in the art. Numerous specific details are set forth such as examples of specific components, devices, and methods, to provide a thorough understanding of embodiments of the present disclosure. It will be apparent to those skilled in the art that specific details need not be employed, that exemplary embodiments may be embodied in many different forms and that neither should be construed to limit the scope of the disclosure. In some exemplary embodiments, well-known processes, well-known device structures, and well-known technologies are not described in detail.

The terminology used herein is for the purpose of describing particular exemplary embodiments only and is not intended to be limiting. As used herein, the singular forms “a”, “an” and “the” may be intended to include the plural forms as well, unless the context clearly indicates otherwise. The terms “comprises,” “comprising,” “including,” and “having,” are inclusive and therefore specify the presence of stated features, integers, steps, operations, elements, and/or components, but do not preclude the presence or addition of one or more other features, integers, steps, operations, elements, components, and/or groups thereof. The method steps, processes, and operations described herein are not to be construed as necessarily requiring their performance in the particular order discussed or illustrated, unless specifically identified as an order of performance. It is also to be understood that additional or alternative steps may be employed.

When an element or layer is referred to as being “on”, “engaged to”, “connected to” or “coupled to” another element or layer, it may be directly on, engaged, connected or coupled to the other element or layer, or intervening elements or layers may be present. In contrast, when an element is referred to as being “directly on,” “directly engaged to”, “directly connected to” or “directly coupled to” another element or layer, there may be no intervening elements or layers present. Other words used to describe the relationship between elements should be interpreted in a like fashion (e.g., “between” versus “directly between,” “adjacent” versus “directly adjacent,” etc.). As used herein, the term “and/or” includes any and all combinations of one or more of the associated listed items.

Although the terms first, second, third, etc. may be used herein to describe various elements, components, regions, layers and/or sections, these elements, components, regions, layers and/or sections should not be limited by these terms. These terms may be only used to distinguish one element, component, region, layer or section from another region, layer or section. Terms such as “first,” “second,” and other numerical terms when used herein do not imply a sequence or order unless clearly indicated by the context. Thus, a first element, component, region, layer or section discussed below could be termed a second element, component, region, layer or section without departing from the teachings of the exemplary embodiments.

Spatially relative terms, such as “inner,” “outer,” “beneath”, “below”, “lower”, “above”, “upper” and the like, may be used herein for ease of description to describe one element or feature's relationship to another element(s) or feature(s) as illustrated in the figures. Spatially relative terms may be intended to encompass different orientations of the device in use or operation in addition to the orientation depicted in the figures. For example, if the device in the figures is turned over, elements described as “below” or “beneath” other elements or features would then be oriented “above” the other elements or features. Thus, the example term “below” can encompass both an orientation of above and below. The device may be otherwise oriented (rotated 90 degrees or at other orientations) and the spatially relative descriptors used herein interpreted accordingly.

What is claimed is:

1. A method of tracking a medical device in a body comprising:
inserting the medical device into a body;
emitting a sonic signal from a transceiver coupled to the medical device;
receiving an echo of the sonic signal with the transceiver; and
detecting a location of the medical device in the body based on the received echo.

2. The method of claim 1, wherein the transceiver is at a predetermined distance from a leading end of the medical device, and wherein detecting a location of the medical device includes detecting a location of the leading end of the medical device.

3. The method of claim 1, further comprising detecting a depth of the medical device based on a reflection of the sonic signal from at least one of a lung and a skin-air interface.

4. The method of claim 1, further comprising detecting a distance of the medical device from a predetermined target location based on a reflection of the sonic signal from a spinal column.

5. The method of claim 1, further comprising distinguishing between a plurality of echoes to detect the location of the medical device.

6. The method of claim 1, wherein emitting a sonic signal comprises emitting an ultrasonic signal.

7. The method of claim 1, wherein the medical device is one of a tunneling tool, a catheter, a neurological stimulating device, a neurological recording device, a shunt, and an electrical lead.

8. A method of creating a subcutaneous tunnel in a body comprising:
inserting a leading end of a tunneling tool into a body;
creating the subcutaneous tunnel as the leading end advances in the body;
emitting a sonic signal from an emitter on the tunneling tool;
receiving an echo of the sonic signal; and
detecting a location of the leading end in the body based on the received echo.

9. The method of claim 8, further comprising detecting a depth of the leading end within the body based on a reflection of the sonic signal from at least one of a lung and a skin-air interface.

10. The method of claim 8, further comprising detecting a distance of the leading end from a predetermined target location based on a reflection of the sonic signal from a spinal column.

11. The method of claim 8, further comprising emitting an external sonic signal from outside the body, receiving the external sonic signal, and detecting a distance of the leading end from a predetermined target location based on the received external sonic signal.

12. The method of claim 8, further comprising distinguishing between a plurality of echoes to detect the location of the leading end.

13. The method of claim 8, further comprising inserting a medical device into the body via the subcutaneous tunnel.

14. The method of claim 8, wherein emitting a sonic signal comprises emitting an ultrasonic signal.

15. A tracking system comprising:
a medical device for being introduced into a body of a patient;
a transceiver that emits a sonic signal and that receives an echo of the sonic signal, the transceiver being coupled to the medical device; and
a controller that detects a location of the medical device in the body based on the echo received by the transceiver.

16. The tracking system of claim 15, wherein the transceiver is an ultrasonic transceiver that emits an ultrasonic signal.

17. The tracking system of claim 15, wherein the controller distinguishes between a plurality of echoes to detect the location of the medical device.

18. The tracking system of claim 15, wherein the medical device includes a leading end, wherein the transceiver is at a predetermined distance from the leading end, and wherein the controller detects a location of the leading end of the medical device based on the echo received by the transceiver.

19. The tracking system of claim 15, wherein the controller detects a depth of the medical device within the body based on a reflection of the sonic signal from at least one of a lung and a skin-air interface.

20. The tracking system of claim 15, wherein the controller detects a distance of the medical device from a predetermined target location based on a reflection of the sonic signal from a spinal column.

21. The tracking system of claim 15, wherein the medical device is one of a tunneling tool, a catheter, a neurological stimulating device, a neurological recording device, a shunt, and an electrical lead.

22. A tunneling system for creating a subcutaneous tunnel in a body comprising:
a tunneling tool having a leading end that creates the subcutaneous tunnel as the leading end advances in the body;
an emitter that emits a sonic signal, the emitter being coupled to the tunneling tool;
a receiver that receives an echo of the sonic signal; and
a controller that detects a location of the leading end in the body based on the echo received by the receiver.

23. The tunneling system of claim 22, wherein the emitter is an ultrasonic emitter that emits an ultrasonic signal.

24. The tunneling system of claim 22, wherein the controller distinguishes between a plurality of echoes to detect the location of the leading end.

25. The tunneling system of claim 22, wherein the controller detects a depth of the leading end within the body based on a reflection of the sonic signal from at least one of a lung and a skin-air interface.

26. The tunneling system of claim 22, wherein the controller detects a distance of the leading end from a predetermined target location based on a reflection of the sonic signal from a spinal column.

27. The tunneling system of claim 22, further comprising an external sonic device that emits a sonic signal from outside the body, and wherein the controller detects a distance of the leading end from a predetermined target location based on the sonic signal from the external sonic device.

28. The tunneling system of claim 22, wherein the tunneling tool is elongate.

29. The tunneling system of claim 22, wherein the tunneling tool includes an attachment mechanism for removably coupling to a medical device.

30. The tunneling system of claim 29, wherein the attachment mechanism includes a hook that is movable relative to the main body.

31. The tunneling system of claim 30, wherein the tunneling tool includes an actuator for selectively moving the hook relative to the main body.

32. A method of creating a subcutaneous tunnel in a body comprising:
coupling an electrical lead of a cardiac defibrillator to a tunneling tool;
inserting a leading end of the tunneling tool into a body;
creating the subcutaneous tunnel as the leading end advances in the body;
advancing the electrical lead in the tunnel;
emitting at least one ultrasonic signal from an emitter coupled adjacent the leading end;
receiving a plurality of echoes of the at least one ultrasonic signal;
detecting a depth of the leading end within the body based on a reflection of the at least one ultrasonic signal from a lung and a skin-air interface;
detecting a distance of the leading end from a predetermined target location based on a reflection of the at least one ultrasonic signal from a spinal column;
detaching the tunneling tool from the electrical lead when the leading end is located approximately at the predetermined target location; and
removing the tunneling tool from the body.
